(19) United States
(12) Patent Application Publication
(10) Pub. No.: US 2021/0186142 A1
(43) Pub. Date: Jun. 24, 2021

(54) Shoe with warming device

(71) Applicant: Hsao-Hsing Chiu, Taipei (TW)

(72) Inventor: Hsao-Hsing Chiu, Taipei (TW)

(21) Appl. No.: 17/195,672

(22) Filed: Mar. 9, 2021

(51) Int. Cl. A43B 7/04; A61F 7/08; A43B 7/06

(52) U.S. Cl. CPC A43B 7/04; A61F 2007/0045; A43B 7/06; A61F 7/08

(57) ABSTRACT

A shoe includes a sole including front and rear compartments; a warming device in the front compartment; a main battery and circuit board in the rear compartment and enclosed by a first insulation member; a switch on a heel cap of the shoe; a display member on the heel cap a light emitting member on the heel cap; and an auxiliary battery enclosed by a second enclosure in the heel cap. The main battery and circuit board is electrically connected to the warming device, the push-button switch, the light emitting member, and the auxiliary battery respectively.

### BACKGROUND OF THE INVENTION

### 1. Field of the Invention

The invention relates to shoes and more particularly to such a pair of shoes having a warming device.

### 2. Description of Related Art

Shoes with ventilation system are well known. Shoes with massaging effect are also well known. Shoes with warming effect are also well known. U.S. Pat. No. 7,347,831 to Chiu discloses a ventilated shoe comprising an outsole; an insole including projections; and a space defined by the insole and the outsole and including resilient pillars in the instep and interconnected the insole and the outsole, a battery compartment, second compartments, motor-driven vibrating devices located in the second compartments, an disposed first switch, a heating element, an disposed second switch, and a circuit board with the first and second switches provided on its edge. Turning on the first switch will activate the vibrating devices to vibrate the projections to perform a first operation to massage the sole. Turning on the second switch will activate the heating element to perform a second operation to warm the foot.

However, the space for installing a battery of the patented shoe is small, i.e., the capacity of the battery is limited. Thus, the patented shoe cannot provide sufficient heat to warm the foot in an extreme cold weather and is quickly consumed.

Thus, the need for improvement still exists.

### SUMMARY OF THE INVENTION

It is therefore one object of the invention to provide a shoe comprising a sole including a front compartment and a rear compartment; a warming device disposed in the front compartment; a main battery and circuit board disposed in the rear compartment and enclosed by a first insulation member; a switch disposed on a heel cap of the shoe; a light emitting member disposed on the heel cap; and an auxiliary battery enclosed by a second enclosure in the heel cap; wherein the main battery and circuit board is electrically connected to the warming device via a first flexible cable, electrically connected to the push-button switch via a second flexible cable, electrically connected to the light emitting member via the second flexible cable, and electrically connected to the auxiliary battery via a third flexible cable.

The above and other objects, features and advantages of the invention will become apparent from the following detailed description taken with the accompanying drawings.

### BRIEF DESCRIPTION OF THE DRAWINGS

FIG. 1 is a perspective view of a shoe according to the invention;

FIG. 2 is an exploded view of the sole, the warming device and the main battery;

FIG. 3 is an exploded view of the shoe;

FIG. 4 schematically depicts the shoe;

FIG. 5 schematically depicts the warming device operated by a mobile phone; and

FIG. 6 is a detailed view of the area in the upper circle of FIG. 4.

### DETAILED DESCRIPTION OF THE INVENTION

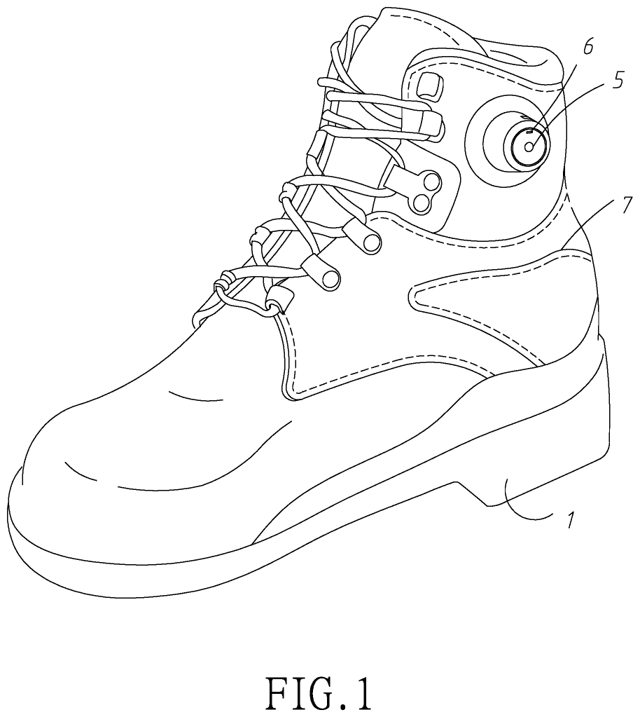

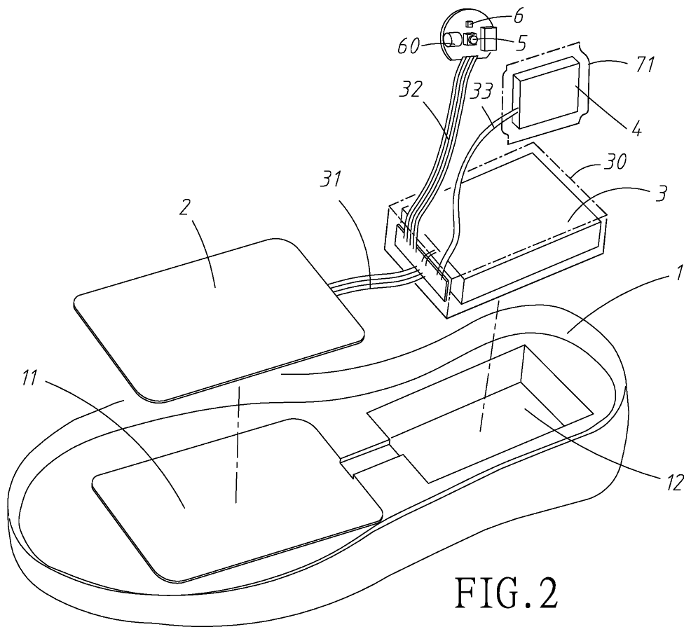

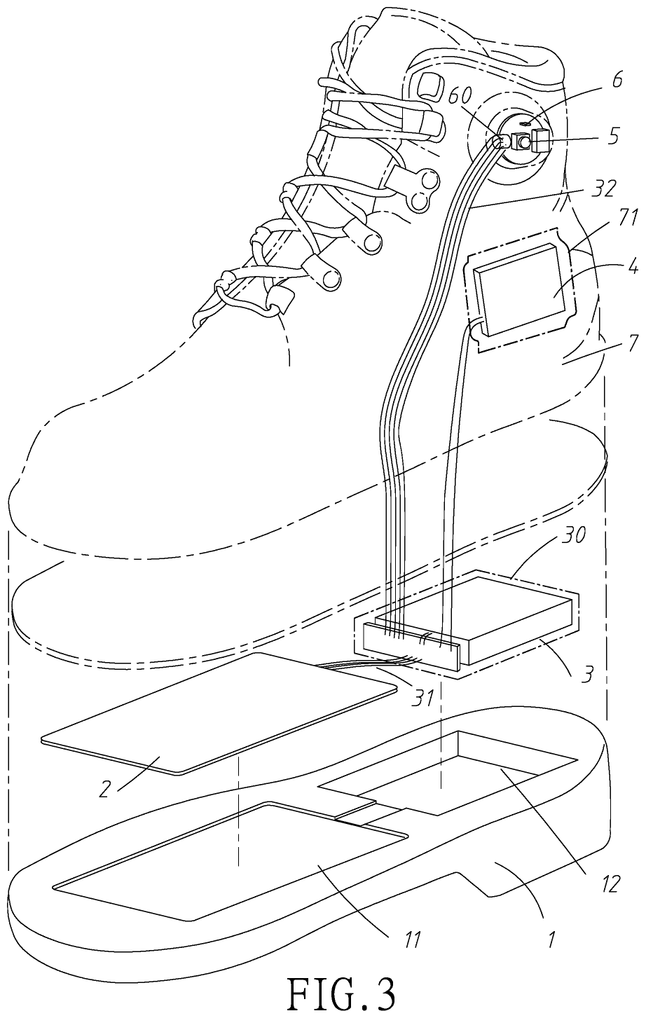

Referring to FIGS. 1 to 6, a shoe in accordance with the invention comprises the following components as discussed in detail below.

A sole 1 includes a front compartment 11 and a rear compartment 12. A warming device 2 is disposed in the front compartment 11 and formed of heating elements. A main battery and circuit board 3 is disposed in the rear compartment 12 and is enclosed by a first insulation member 30. The main battery and circuit board 3 is electrically connected to the warming device 2 via a first flexible cable 31. The main battery and circuit board 3 is also electrically connected to a push-button switch 5 disposed on a heel cap 7, a display member 6 disposed on the heel cap 7 for showing on or off of the push-button switch 5, and a light emitting member (e.g., light-emitting diode (LED)) 60 disposed on the heel cap 7 via a second flexible cable 32. The main battery and circuit board 3 is further electrically connected to an auxiliary battery 4 enclosed by a second enclosure 71 in the heel cap 7 via a third flexible cable 33. It is noted that both the main battery and circuit board 3 and the auxiliary battery 4 are capable of withstanding low temperature in cold weather.

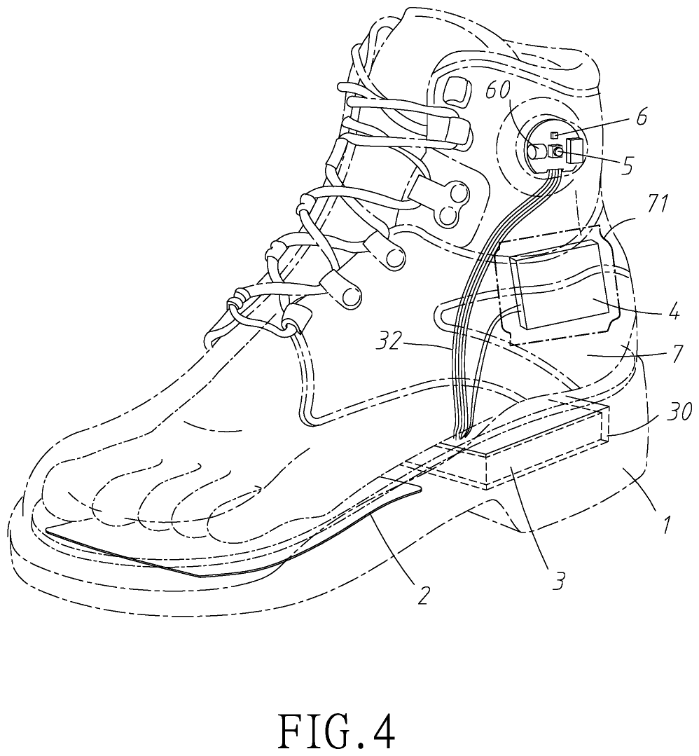

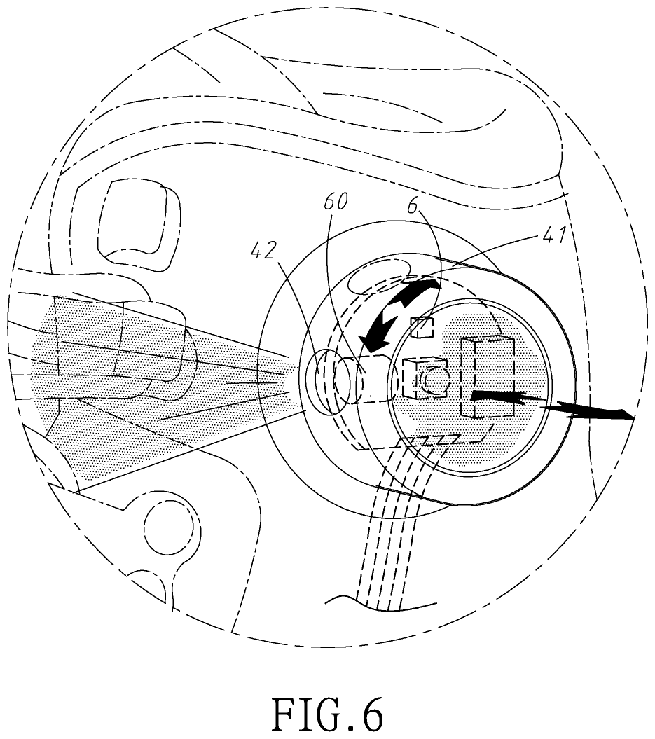

As shown in FIGS. 4 and 6 specifically, in use, a wearer may press the push-button switch 5 for 3 seconds to activate the main battery and circuit board 3 which in turn supplies electricity to the warming device 2 to warm the shoe (i.e., warming). At this time, the light emitting member 60 emits red light and the display member 6 shows the on state of the push-button switch 5 in which the internal temperature of the shoe reaches 60-65° C. within 6 minutes. The light emitting member 60 emits violet light when the internal temperature of the shoe lowers to 40-45° C. The light emitting member 60 emits blue light when the internal temperature of the shoe lowers to 35-40° C. The wearer is aware of the internal temperature of the shoe by seeing the color of the light emitting member 60. The wearer may press the push-button switch 5 for 3 seconds to activate the main battery and circuit board 3 again if the light emitting member 60 emits blue light.

Also, the auxiliary battery 4 is activated to prolong the time of supplying electricity to the warming device 2. Illumination provided by the light emitting member 60 is for the purpose of safety device and is useful especially in a dark environment or night time.

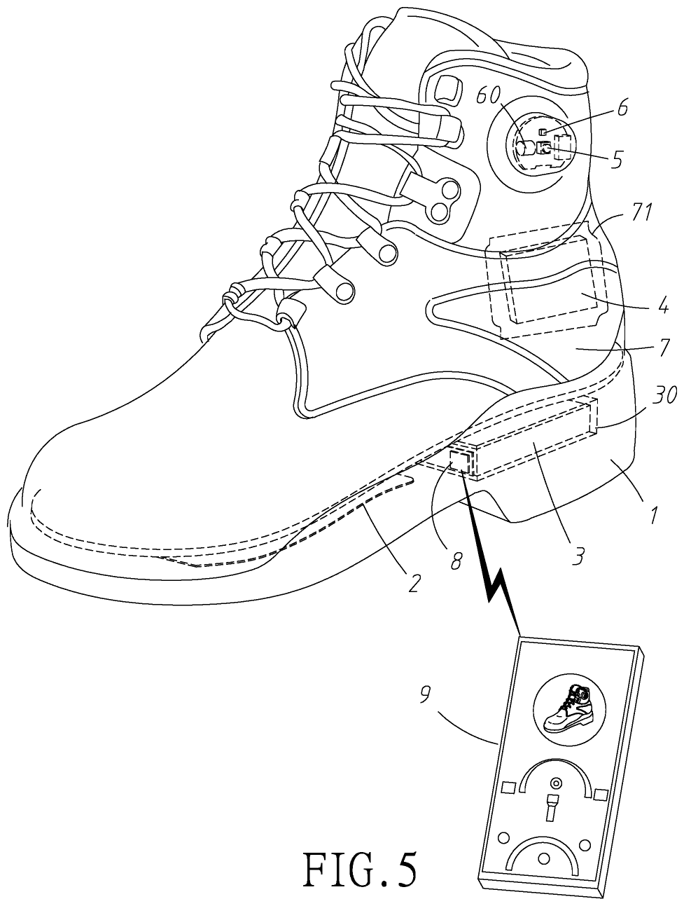

As shown in FIG. 5 specifically, a receiving member 8 is mounted in the sole 1 adjacent to the main battery and circuit board 3 and configured to receive wireless signals of radio frequency (e.g., Bluetooth® signals) transmitted from APP of a mobile phone 9. That is, a person may use the mobile phone 9 to activate a chip in the receiving member 8 which in turn activates the main battery and circuit board 3. Thus, the person can control power of the shoe, adjust temperature of the shoe and cause the shoe to emit light in the night time. This is particularly applicable to senior persons.

While the invention has been described in terms of preferred embodiments, those skilled in the art will recognize that the invention can be practiced with modifications within the spirit and scope of the appended claims.

What is claimed is:

1. A shoe, comprising:
a sole including a front compartment and a rear compartment;
a warming device disposed in the front compartment;
a main battery and circuit board disposed in the rear compartment and enclosed by a first insulation member;
a switch disposed on a heel cap of the shoe;
a display member disposed on the heel cap;
a light emitting member disposed on the heel cap; and
an auxiliary battery enclosed by a second enclosure in the heel cap;
wherein the main battery and circuit board is electrically connected to the warming device via a first flexible cable, electrically connected to the push-button switch via a second flexible cable, electrically connected to the light emitting member via the second flexible cable, electrically connected to the display member via the second flexible cable, and electrically connected to the auxiliary battery via a third flexible cable.

2. The shoe of claim 1, further comprising a receiving member disposed in the sole adjacent to the main battery and circuit board and configured to receive wireless signals of radio frequency from a mobile phone so that a user may perform an APP of the mobile phone to control on or off of the switch, internal temperature of the shoe, and on or off of the light emitting member.
